SEQUENCE LISTING (11 sequences)

SEQ ID NO: 1 - DNA, 23 bp, Artificial Sequence
  nucleotides binding Smad gene
ggtgtctagacatagtctagaca

SEQ ID NO: 2 - DNA, 22 bp, Artificial Sequence
  alpha-SMA-S
gcccagccaagcactgtcagga

SEQ ID NO: 3 - DNA, 23 bp, Artificial Sequence
  alpha-SMA-AS
tcccaccatcaccccctgatgtc

SEQ ID NO: 4 - DNA, 19 bp, Artificial Sequence
  CTGF-S
ggcttaccgactggaagac

SEQ ID NO: 5 - DNA, 18 bp, Artificial Sequence
  CTGF-AS
aggaggcgttgtcattgg

SEQ ID NO: 6 - DNA, 20 bp, Artificial Sequence
  Fibronectin-S
gtggctgaagacacaaggaa

SEQ ID NO: 7 - DNA, 19 bp, Artificial Sequence
  Fibronectin-AS
cctgccattgtaggtgaat

SEQ ID NO: 8 - DNA, 20 bp, Artificial Sequence
  NOX4-S
cacctctgcctgttcatctg

SEQ ID NO: 9 - DNA, 21 bp, Artificial Sequence
  NOX4-AS
ggctctgcttagacacaatcc

SEQ ID NO: 10 - DNA, 22 bp, Artificial Sequence
  GAPDH-S
gtctcctctgacttcaacagcg

SEQ ID NO: 11 - DNA, 22 bp, Artificial Sequence
  GAPDH-AS
accaccctgttgctgtagccaa

(19) United States
(12) Patent Application Publication
(10) Pub. No.: US 2023/0015532 A1
(43) Pub. Date: Jan. 19, 2023

(54) PHARMACEUTICAL COMPOSITION, FOR PREVENTING OR TREATING FIBROSIS, COMPRISING PHEOPHORBIDE COMPOUND AS ACTIVE INGREDIENT

(71) Applicant: PAEAN BIOTECHNOLOGY INC., Daejeon (KR)

(72) Inventors: Chun-Hyung KIM, Sejong (KR); Shin-Hye YU, Seoul (KR); Seo-Eun LEE, Suwon-si, Gyeonggi-do (KR); Jonghyeok PARK, Seoul (KR); Sunyeou KIM, Seoul (KR); Amna PARVEEN, Daejeon (KR); Kyuboem HAN, Daejeon (KR)

(21) Appl. No.: 17/780,936

(22) PCT Filed: Dec. 11, 2020
(86) PCT No.: PCT/KR2020/018167

§ 371 Date: May 27, 2022

(30) Foreign Application Priority Data
Dec. 11, 2019 (KR) 10-2019-0164277

(51) Int. Cl. A61K 31/409; A23L 33/10; A61P 11/00

(52) U.S. Cl. CPC A61K 31/409; A23L 33/10; A61P 11/00

(57) ABSTRACT

The present invention relates to a pharmaceutical composition for preventing or treating fibrosis, containing a pheophorbide compound as an active ingredient. The pharmaceutical composition can effectively inhibit fibrosis of tissues by inhibiting signaling of TGF-β which causes fibrosis and inhibiting activation and expression of collagen and fibronectin, and is significantly superior in the anti-fibrotic activity compared to nintedanib or pirfenidone, which are commercially available therapeutic agents for pulmonary fibrosis, and therefore may be widely used in the prevention or treatment of fibrosis.

### TECHNICAL FIELD

The present invention relates to a pharmaceutical composition for preventing or treating fibrosis, comprising a pheophorbide compound as an active ingredient.

### BACKGROUND ART

Fibrotic diseases occupying about 45% of the cause of death in the Western society belong to a serious disease field where the prophylactic treatment is limited since they can be diagnosed after significant clinical progression of the disease, and no effective therapeutic means to directly resolve the progressive and pre-existing fibrosis are available to date.

Myofibroblasts which are abnormally activated fibroblasts, are reported as causative cells that govern the progression of fibrotic diseases. Myofibroblasts are characterized by causing the accumulation in tissues of extracellular matrix (ECM) consisting of fibrous type 1,3 collagen and fibronectin and the inactivation of enzyme genes that degrade the extracellular matrix.

It is known that growth factors such as TGF-β1 (transforming growth factor-beta 1), TNF-α (tumor necrosis factor-alpha), IL-1 (interleukin-1), IL-6, IL-13, PDGF (platelet-derived growth factor), etc., which are secreted from damaged epithelium, endothelium, bone marrow-derived fibrin cells, immune cells, and the like, and cytokines are involved in differentiation and activation of myofibroblasts.

Accordingly, blocking the activation and signaling of the growth factors and cytokines that exacerbate fibrosis can be a starting point for treating fibrosis, and drugs targeting TGF-β1, IL-13, CTGF(connective-tissue growth factor), PDGF, αvβ6 integrin, galectin-3, LOXL2 (lysyl oxidase homolog 2), transglutaminase-2, NOX4 (NADPH oxidase 4) or JNK (Jun N-terminal kinases) inhibitors are being developed in the art for treating fibrotic diseases.

Currently, drugs administered to patients with idiopathic pulmonary fibrosis use steroids and immunosuppressive agents alone or in combination. Although these drugs exhibit a therapeutic efficacy in about 10-30% of the total patients, their use is gradually on the decline due to the median survival rate of less than 3 years, which is not effective, and many side effects.

On the other hand, pirfenidone, a drug that inhibits the promoter response of TGF-β, was recommended for conditional prohibition of use in the 2011 guidelines. However, it has recently been updated to a conditionally usable drug and has been shown to reduce the spread of myofibroblasts in the animal study. However, the subsequent clinical tests reported no marked effects and the occurrence of side effects such as nausea, vomiting, dyspepsia and the like.

In addition, nintedanib, a tyrosine kinase inhibitor, is a drug that inhibits growth factor receptors of various cells and is known to inhibit the action of cytokines that promote fibrosis at the cell level, reduce collagen synthesis by TGF-β, and alleviate pulmonary fibrosis in fibrosis-induced animal experiments. It has shown the effect of delaying the reduction of lung function in a wide range of patient groups in clinical trials, but it is known to have no ability to treat pulmonary fibrosis.

As such, pirfenidone and nintedanib focus on the delay of reduction of lung function and have a limitation that they do not function as drugs that stop the progression of fibrosis itself. Accordingly, the present inventors have made extensive efforts to develop an agent effective for preventing or treating fibrosis by inhibiting fibrosis of tissues. As a result, the present inventors have completed the present invention by confirming that pheophorbide a inhibits TGF-β signaling and efficiently inhibits activation of extracellular matrix collagen and fibronectin, which are key proteins of fibrotic diseases.

### DETAILED DESCRIPTION OF THE INVENTION

### Technical Problem

It is an object of the present invention to provide a pharmaceutical composition for preventing or treating fibrosis, which contains, as an active ingredient, a compound for effectively preventing and treating fibrosis by inhibiting fibrosis of tissues.

Another object of the present invention is to provide a food composition for preventing or ameliorating fibrosis, containing the above compound as an active ingredient.

### Solution to Problem

In order to achieve the above object, the present invention provides a pharmaceutical composition for preventing or treating fibrosis, which contains a pheophorbide compound as an active ingredient.

The present invention also provides a food composition for preventing or ameliorating fibrosis, containing a pheophorbide compound as an active ingredient.

### Effect of the Invention

The pharmaceutical composition for preventing or treating fibrosis according to the present invention can effectively inhibit fibrosis of tissues by inhibiting TGF-β signaling that causes fibrosis and inhibiting activation and expression of collagen and fibronectin. In addition, since it shows an anti-fibrotic activity remarkably superior to nintedanib or pirfenidone, which is a commercially available therapeutic agent for pulmonary fibrosis, it may be useful for prevention or treatment of fibrosis.

### BRIEF DESCRIPTION OF DRAWINGS

FIG. 1 shows the process of separating the pheophorbide a from the Dendropanax Morbiferus.

FIG. 2 is the schematic diagram of an experiment for analyzing the anti-fibrotic activity of pheophorbide a at the cell level.

FIG. 3 is the experimental result of the cytotoxicity of pheophorbide a in CCD8-Lu and LL-29 which are human lung fibroblasts.

FIG. 4 is the result of a luciferase assay for determining the difference in DNA binding capacity of Smad proteins according to treatment with TGF-β and/or pheophorbide a.

FIG. 5 is the result of comparing the difference in the expression level of collagen 1A, fibronectin and alpha-smooth muscle actin proteins by treating CCD8-Lu cells which are human lung fibroblasts with different concentrations of TGF-β and/or pheophorbide a, using Western blotting.

FIG. 6 is the result of comparing the difference in the expression level of collagen 1A by treating CCD8-Lu cells which are human lung fibroblasts with different concentrations of TGF-β and/or pheophorbide a, using an immunocytochemistry analysis.

FIGS. 7 to 10 are the results of comparing the difference in the expression level of α-SMA gene (FIG. 7), CTGF gene (FIG. 8), fibronectin gene (FIG. 9), and NOX4 gene (FIG. 10) by treating CCD8-Lu cells which are human lung fibroblasts with TGF-β, and pheophorbide a, nintedanib, or pirfenidone, using quantitative polymerase chain reaction (qRT-PCR).

FIG. 11 is the result of comparing the difference in the expression level of collagen 1A, fibronectin and alpha-smooth muscle actin proteins by treating CCD8-Lu cells which are human lung fibroblasts with TGF-β, and pheophorbide a, nintedanib, or pirfenidone, using Western blotting.

FIG. 12 is the result of comparing the difference in the expression level of collagen 1A proteins by treating CCD8-Lu cells which are human lung fibroblasts with TGF-β, and pheophorbide a or nintedanib, using an immunocytochemistry analysis.

FIG. 13 is the result of comparing the difference in the expression level of Smad, phosphorylated Smad3, ERK and phosphorylated ERK proteins by treating CCD8-Lu cells which are human lung fibroblasts with TGF-β, and pheophorbide a or nintedanib, using Western blotting.

### BEST MODE FOR CARRYING OUT THE INVENTION

### Pharmaceutical Composition for Preventing or Treating Fibrosis

One aspect of the present invention provides a pharmaceutical composition for preventing or treating fibrosis, which comprises a pheophorbide compound as an active ingredient.

In the present invention, the term “pheophorbide compound” has the structure of the following formula:

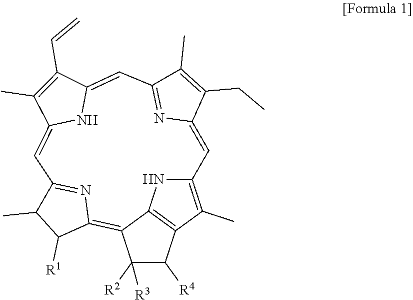

wherein R¹, R² and R³ are each any one selected from the group consisting of H, C₁-C₄ straight or branched alkyl, carboxyl, C₁-C₄ alkoxycarbonyl, C₁-C₄ alkoxycarbonyl C₁-C₄ alkyl, amino, amino-C₁-C₄-alkyl and oxo (═O); and

R⁴ is any one selected from the group consisting of H, hydroxy, oxo(═O), C₁-C₄ straight or branched alkyl, C₁-C₄ straight or branched alkoxy, amino, and amino-C₁-C₄ alkyl.

In the present invention, the pheophorbide compound may be pheophorbide a, but is not limited thereto.

In the present invention, the term “pheophorbide a” is a compound represented by the molecular formula of C₃₅H₃₆N₄O₅ and has the structure of the following formula:

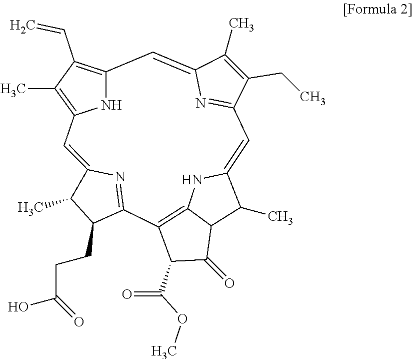 [SMILES: C=Cc1c(C)c2cc3nc(c4c5[nH]c(cc6nc(cc1[nH]2)C(C)=C6CC)C(C)C5C(=O)[C@@H]4C(=O)OC)[C@@H](CCC(=O)O)[C@@H]3C]

The pheophorbide compound, specifically pheophorbide a, can inhibit TGF-β signaling and thereby inhibit differentiation and activation of myofibroblasts. Thus, the compound can inhibit the expression of α-SMA, which is a major marker of myofibroblasts, as well as the expression of collagen and fibronectin, which are extracellular matrix proteins characteristic of fibrosis.

In the present invention, the term “TGF-β” refers to a key cytokine that initiates and terminates repair of damaged tissue, and continued production of TGF-β results in fibrosis of the tissue. In particular, the expression of TGF-β1 mRNA in liver tissue findings of chronic liver diseases is closely related to the expression of collagen I mRNA. In addition, TGF-β1 protein is expressed only in a region where fibrosis has progressed, and is not expressed in normal liver tissues or inactive regions. Therefore, it is known that TGF-β plays an important role in hepatic fibrosis and cirrhosis.

Furthermore, it is known that TGF-β is involved in differentiation and activation of myofibroblasts which are major causative cells of fibrosis. In the activation of the fibrosis process, the key of the molecular mechanism is the activation of myofibroblasts by TGF-β and Smad dependent signaling. Binding of TGF-β1 to the TGF-β1 receptor present in the cell membrane results in the transmission of signals of the specific function of TGF-β1. In addition, the activated TGF-β1 receptor induces phosphorylation reaction of Smad2 and Smad3 proteins, and phosphorylated Smad2/Smad3 binds to Smad4 proteins to form complexes. The Smad complex is known to translocate into the cell nucleus and cause transcription of proteins that constitute extracellular matrices such as fibronectin and collagen.

Therefore, the pheophorbide compound, particularly pheophorbide a, can be characterized by inhibiting TGF-β signaling and inhibiting the phosphorylation of Smad proteins, thereby inhibiting the expression of fibronectin and collagen constituting the extracellular matrix.

As used herein, the term “fibrosis” means that excess fibrous connective tissue is formed in an organ or tissue. It can be distinguished from fibrous tissue as a normal component in organs or tissues. Fibrosis can be understood as a fatal disease in which extracellular matrix such as fibronectin, collagen, etc., accumulated in excess by fibroblasts, thereby resulting in a sustained loss of human tissue function due to hardening of organ tissues.

The fibrosis may occur, for example, in any one or more selected from the group consisting of kidney, liver, lung, skin, heart, pancreas, urinary system, reproductive system, sweat gland, nerve, brain, bone marrow, muscle, and joint.

In the present invention, the fibrosis may be any one or more selected from the group consisting of, but not limited to, hepatic fibrosis, pulmonary fibrosis, skin fibrosis, arthrofibrosis, nerve fibrosis, pancreatic fibrosis, kidney fibrosis, muscle fibrosis and peritoneal fibrosis.

More specifically, the fibrosis may be any one or more selected from the group consisting of, but not limited to, pulmonary fibrosis, idiopathic pulmonary fibrosis, radiation-induced lung injury or pulmonary fibrosis, pulmonary edema, cystic fibrosis, hepatic fibrosis, endomyocardial fibrosis, myocardial infarction, artrial fibrosis, glial scar, renal fibrosis, myelofibrosis, arthrofibrosis, fat fibrosis, skin fibrosis, nerve fibrosis and muscle fibrosis.

As used herein, the term “hepatic fibrosis” refers to a symptom of proliferation of fibrous tissue caused by chronic damage of the liver and may include, but is not limited to, the one due to any one or more selected from the group consisting of chronic liver disease, hepatitis B virus infection, hepatitis C virus infection, hepatitis D virus infection, schistosomiasis, alcoholic liver disease or non-alcoholic steatohepatitis, metabolic disease, protein deficiency, coronary artery disease, autoimmune hepatitis, cystic fibrosis, alpha-1 antitrypsin deficiency, primary biliary cirrhosis, drug response and toxin.

Liver fibrosis is a pre-disease of liver cirrhosis and begins by the action of various cytokines and growth factors as a result of severe liver injury leading to chronic liver disease. In general, hepatic fibrosis consists of reversible, thin fibrils, and if no nodules are formed and the cause of liver injury is temporary, increased extracellular matrix (ECM) is degraded by apoptosis and matrix metalloproteinases (MMPs) to allow normal recovery. However, repeated continuation of the hepatic fibrosis process results in the formation of thick fibrils and progression to nodal cirrhosis. In addition, liver cirrhosis is induced through the process of hepatic fibrosis in which hepatocytes are damaged by various inflammation-inducing factors and abnormal extracellular matrix proteins including collagen are accumulated. Thus, it is important to control the accumulation of extracellular matrix for the regulation of expression of liver cirrhosis. Inflammation reactions in the event of damage to hepatocytes activate hepatic stellate cells in the resting phase to secrete extracellular matrix and various cytokines and chemokines, among which TGF-β1 serves as a potent growth inhibitor. TGF-β1, a 25 kD substance, is secreted in an inactive latent form by binding to a latent TGF-β1 binding protein and is present in a form binding to extracellular matrices such as type 1,4 collagen, laminin and decorin, which is activated by various stimuli. TGF-β1 modulates collagen expression by reducing collagenase production or increasing collagenase inhibitor production, increases production of TNF-α, IL-1, PDGF, etc. in macrophages and plays an important role in the fibrosis process. It is now known that TGF-β1 is expressed only in the region where fibrosis has progressed, not in normal liver tissue or in inactive regions, and plays an important role in hepatic fibrosis.

On the other hand, non-alcoholic fatty liver can be caused by obesity, diabetes, hyperlipidemia, drugs and the like regardless of alcohol drinking, and covers a wide range of diseases including simple fatty liver (steatosis) which does not accompany inflammation reactions, and non-alcoholic steatohepatitis (NASH) showing hepatocellular inflammation, advanced fibrosis and liver cirrhosis, depending on the progression of the disease. It is reported that, with an increase in adult diseases due to high fat and high calorie diet in modern society, 20-30% of adult populations in developed countries exhibit non-alcoholic fatty liver diseases (NAFLD), among which 2-3% are progressed to non-alcoholic steatohepatitis (NASH) patients who particularly exhibit steatohepatitis findings accompanied by fibrosis and inflammation histologically and have the high risk of developing liver cirrhosis, liver failure and liver cancer.

The term “pulmonary fibrosis” as used herein refers to the development of scarred (fibrous) tissue due to the formation or development of excessive fibrous connective tissue in the lungs (fibrosis). Specifically, pulmonary fibrosis is a chronic disease that causes swelling and scarring of alveoli and interstitial tissues of the lungs. Such scarred tissue replaces healthy tissue and causes inflammation, and chronic inflammation can be seen as a sign of fibrosis. Such damage to the lung tissue may result in stiffness of the lungs and may make the individual difficult to maintain spontaneous breathing.

Specifically, the pulmonary fibrosis may include, but is not limited to, any one or more selected from the group consisting of idiopathic pulmonary fibrosis; radiation-induced lung injury; nonspecific interstitial pneumonia; acute interstitial pneumonia; cryptogenic organizing pneumonia; respiratory bronchiolitis associated interstitial lung disease; desquamative interstitial pneumonia; lymphoid interstitial pneumonia; interstitial pulmonary fibrosis; and pulmonary fibrosis caused by diffuse pulmonary fibrosis, pulmonary edema, cystic fibrosis, and metabolic diseases.

The term “skin fibrosis” as used herein is excessive scarring of skin, and is a result of a pathological wound healing response. There are a wide range of fibrotic skin diseases: scleroderma, nephrogenic fibrosing dermopathy, mixed connective tissue diseases, scleromyxedema, scleredema, and eosinophilic fasciitis. Exposure to chemicals or physical agents (mechanical trauma, burn wounds) is also potential causes of fibrotic skin diseases. Dermal fibrosis can be driven by immune, autoimmune and inflammatory mechanisms. The balance of collagen production and degradation by fibroblasts plays a critical role in the pathophysiology of dermal fibrosis. Certain cytokines such as transforming growth factor-β (TGF-β) and interleukin-4 (IL-4) promote wound healing and fibrosis. Fibroblasts in normal skin are quiescent. They synthesize the controlled amounts of connective tissue proteins and have low proliferative activity. Following skin injury, these cells become activated, i.e., they express α-smooth muscle actin (α-SMA) and synthesize large amounts of connective tissue proteins. The activated cells are often called as myofibroblasts. Here, “skin fibrosis” may also include “scleroderma”.

As used herein, the term “cardiac fibrosis” refers to the phenomenon in which the heart is hardened due to excessive deposition of matrix proteins between heart cells. The cardiac fibrosis is mainly present in the heart of patients with myocardial infarction and is the main cause of decreased heart function. The cardiac fibrosis may include, but is not limited to, any one or more selected from the group consisting of endocardial fibrosis, atrial fibrosis, heart failure, myocardial infarction, and cardiac fibrosis due to metabolic diseases. In addition, since fibrosis of the heart is a major cause of heart failure and myocardial infarction, the term “cardiac fibrosis” can be interpreted to encompass heart failure and/or myocardial infarction caused by cardiac fibrosis.

In the present invention, the pharmaceutical composition may further contain a pharmaceutically acceptable carrier.

Pharmaceutically acceptable carriers comprised in the pharmaceutical composition of the present invention are those conventionally used in preparation, and include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, mineral oil, saline, phosphate buffered saline (PBS) or medium, and the like, and may further include lubricants, wetting agents, sweeteners, flavoring agents, emulsifiers, suspending agents, preservatives, etc. in addition to the above components.

The pharmaceutical composition of the present invention can be prepared in a unit dosage form by formulating with a pharmaceutically acceptable carrier and/or excipient or by being incorporated into a large-capacity container according to a method readily practiced by persons having ordinary skill in the art, and may further comprise a dispersing agent or a stabilizing agent.

The formulation of the pharmaceutical composition may vary depending on the method of use, but may be prepared as plasters, granules, powders, syrups, solutions, fluidextracts, emulsions, suspensions, infusions, tablets, injections, capsules, and pills. Formulations of the pharmaceutical compositions also include ointments, pastes, creams, lotions, gels, powders, solutions, sprays, inhalants or patches, which are formulations for topical or transdermal administration.

The pharmaceutical composition may be aseptically mixed with a pharmaceutically acceptable carrier and, if necessary, preservatives, buffers, etc. Ointments, pastes, creams and gel formulations according to the present invention may additionally contain, as an excipient, animal and vegetable fats, oils, waxes, paraffins, starches, tragacanth, cellulose derivatives, polyethylene glycols, silicones, bentonites, silicic acid, talc, zinc oxide, or mixtures thereof.

The pharmaceutical composition of the present invention can provide the desired preventive, ameliorative or therapeutic effect of fibrosis when containing an effective amount of pheophorbide a. As used herein, the term “effective amount” refers to an amount that exhibits a more response than a negative control, preferably an amount sufficient to prevent, ameliorate or treat fibrosis. The pharmaceutical composition of the present invention may contain a pheophorbide compound, pheophorbide a in one embodiment, in an amount of 0.01 to 99.9%, and the balance may be occupied by a pharmaceutically acceptable carrier. The compound comprised in the pharmaceutical composition of the present invention will vary depending on the form in which the composition is commercialized, and the like.

The total effective amount of the pharmaceutical composition of the present invention can be administered to a patient in a single dose, or by a fractionated treatment protocol in which multiple doses are administered over a long period of time. The pharmaceutical composition of the present invention can vary the content of the active ingredient depending on the degree of the disease. For example, it may be administered in one to several divided doses such that it is administered in an amount of preferably 0.001 ug to 100 mg, more preferably 0.01 μg to 10 mg, per kg body weight per day on the basis of pheophorbide a. However, since the dose of pheophorbide a is determined by considering various factors such as age, body weight, health condition, sex, severity of disease, diet and excretion rate of the patient as well as the administration route of the pharmaceutical composition and the frequency of the treatment. Considering the above, persons having ordinary skill in the art will be able to determine the appropriate effective dose of pheophorbide a according to the specific use for preventing, treating or ameliorating fibrosis. The pharmaceutical composition according to the present invention is not particularly limited in its formulation, route of administration and method of administration as long as the effect of the present invention is achieved.

### Food Composition for Preventing or Ameliorating Fibrosis

According to another aspect of the present invention, there is provided a food composition for preventing or ameliorating fibrosis, containing a pheophorbide compound as an active ingredient.

In the present invention, the pheophorbide compound may be, but not limited to, pheophorbide a.

In the present invention, the term “food” means a natural product or a processed product containing one or more nutrients, and preferably means a product in a state of being directly edible through certain processing steps, and the term includes all of food, food additives, health-functional food, beverages, beverage additives and the like as ordinary meanings.

When the composition of the present invention is prepared into a food composition, it contains not only the Dendropanax Morbiferus extract as an active ingredient but also components usually added in the preparation of food, for example, proteins, carbohydrates, fats, nutrients, seasoning agents and flavoring agents.

### Method of Preventing and Treating Fibrosis

Another aspect of the present invention provides a method of preventing and treating fibrosis, comprising administering a pheophorbide compound to a subject.

The subject may be a subject having fibrosis. The subject may also be a mammal, preferably a human. In this case, the pheophorbide compound is as described above, and preferably, the pheophorbide compound may be pheophorbide a. In addition, the route, the dose, and the frequency of administration of the compound may vary depending on the condition of the patient and the presence or absence of side effects, the compound can be administered to a subject in various methods and amounts, and the optimum method of administration, dose and frequency of administration can be selected by persons having ordinary skill in the art within appropriate ranges. The type of fibrosis is also as described above.

### Use of Pheophorbide Compound for Treating Fibrosis

Another aspect of the present invention provides use of a pheophorbide compound for treating fibrosis.

In this case, the pheophorbide compound is as described above, and preferably, the pheophorbide compound may be pheophorbide a. The type of fibrosis is also as described above.

### MODE FOR CARRYING OUT THE INVENTION

Hereinafter, the present invention will be described in more detail by way of examples. However, it will be apparent to persons having ordinary skill in the art that the following examples are only for illustrating the present invention, and the scope of the present invention is not limited thereto.

### Preparation Example 1. Preparation of Dendropanax Morbiferus Extract

Leaves, stems, roots, etc. of Dendropanax Morbiferus were washed and ground using a grinder, and then ethanol or methanol was added to about 8 g of the ground product to be a concentration of 20 w/v %. It was immersed and stirred for at least 4 hours and then filtered to separate a primary liquid component. The separated solids were again immersed and stirred in ethanol or methanol for at least 4 hours and then filtered to obtain a secondary liquid component. The obtained primary liquid component and secondary liquid component were mixed, the mixture was concentrated under reduced pressure, and the residue was freeze-dried to obtain a Dendropanax Morbiferus extract.

### Preparation Example 2. Isolation of Pheophorbide a from Dendropanax Morbiferus Extract

The Dendropanax Morbiferus extract obtained in Preparation Example 1 was fractionated with petroleum ether, hexane, chloroform, dichloromethane, and ethyl acetate in the order as described to obtain fractions. The highly active chloroform fraction was subjected to elution conditions of petroleum ether, ethyl ether, methanol and water on a silica gel column to obtain a total of 6 subfractions (F-1 to F-6). Among them, F-2 fraction was subjected to elution conditions of petroleum ether, ethyl ether and methanol on a silica gel column to obtain a total of 10 subfractions (F-1a to F-10a).

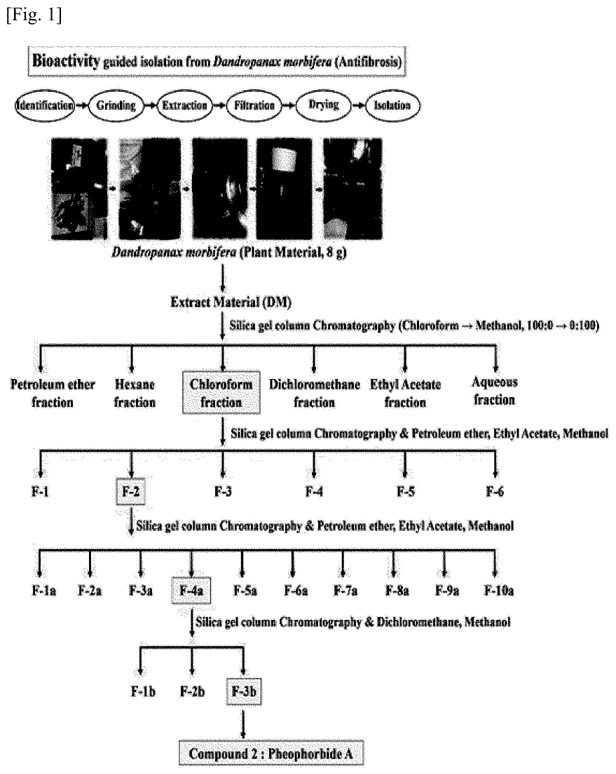

Among them, a total of three subfractions (F-1b to F-3b) were obtained by subjecting the F-4a subfraction to the elution condition of dichloromethane:methano=95:05 (v/v) on a silica gel column. Among them, pheophorbide a was confirmed in the F-3b subfraction by NMR analysis as shown in Table 1 below (FIG. 1).

TABLE 1

| Position | ¹H Chemical shift |
|---|---|
| A | 9.42 |
| β | 9.56 |
| δ | 8.60 |
| 2a | 8.00 |
| 2b | 6.25 |
| 10 | 6.27 |
| 8 | 4.50 |
| 7 | 4.05 |
| 10b | 3.88 |
| 4a | 3.70 |
| 5a | 3.65 |
| 1a | 3.33 |
| 3a | 3.20 |
| 7a, 7b | 2.30-2.50 |
| 8a | 1.80 |
| 4b | 1.65 |

### Experimental Example 1. Experiment for Cytotoxicity of Pheophorbide a

In order to confirm the cytotoxicity of pheophorbide a obtained in Preparation Example 2, CCD8-Lu cells and LL-29 cells which are human lung-derived fibroblasts, were used. CCD8-Lu cells and LL-29 cells were inoculated into 96 well plates with 2×0³ cells/well and stabilized while culturing for 24 hours in a wet CO₂ incubator at 37° C. The cultured cells were treated with different concentrations of pheophorbide a obtained in Preparation Example 2 and, 72 hours later, treated with the WST-1 reagent. After 1 hour, the survival rate of the cells was measured using a microplate reader (EZ Read 400, biochrom, UK) to determine the absorbance at 450 nm, and the toxicity was evaluated.

The cell survival rate as a result of the experiment is shown in FIG. 3 by expressing the percentage of the absorbance of the sample-treated group relative to the control group. As shown in FIG. 3, it was confirmed that pheophorbide a had little cytotoxicity up to a concentration of 2.5 μM and exhibited cytotoxicity of about 25% at a concentration of 5 μM and about 75% at a concentration of 10 μM.

### Experimental Example 2. Confirmation of Inhibitory Effect of Pheophorbide a on TGF-β Signaling Pathway

A vector was prepared in which a gene obtained by repeating eight times the nucleotide sequence (5′-GGTGTCTAGACATAGTCTAGAGACA-3′ SEQ ID NO: 1) to which the Smad gene can bind was linked to a gene encoding luciferase as a reporter gene. The above vector was transfected into Balb/c3T3 cells together with a vector in which the RSV promoter gene was linked to a gene encoding beta-galactosidase. After 6 hours, the negative control group was treated with nothing, the positive control group was treated with TGF-β at a concentration of 5 ng/ml, and the experimental groups were treated with pheophorbide a obtained in Preparation Example 2 at each concentration of 0.06 μM, 0.12 μM, 0.25 μM, 0.5 μM, 1 μM, and 5 μM, together with 5 ng/ml of TGF-β.

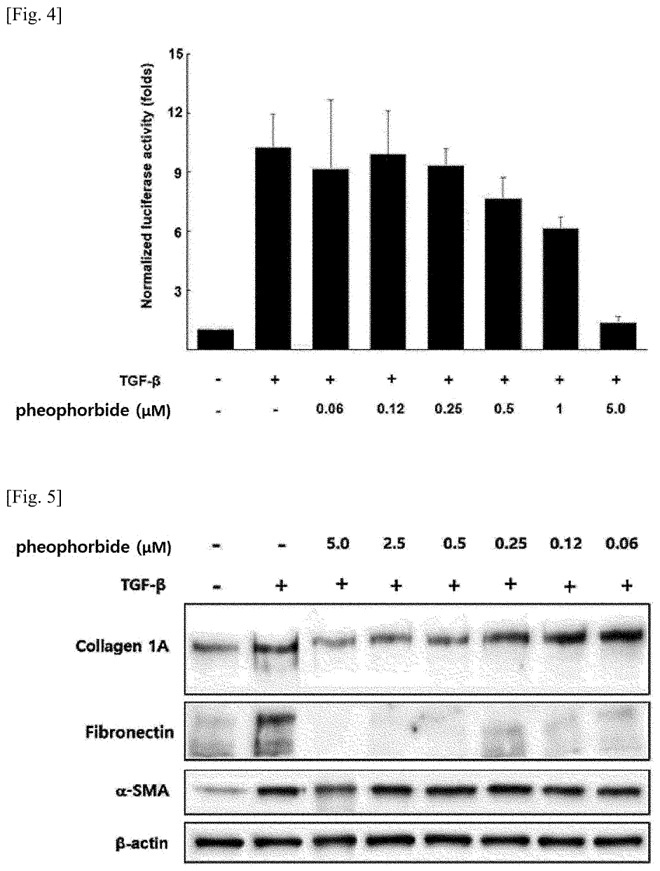

The cells were then cultured in 5% CO₂ incubator at 37° C. for 20 hours, and then luciferase analysis was performed. As a result, as shown in FIG. 4, it was confirmed that the activity of the Smad protein was increased by TGF-β in Balb/c3T3 cells, but the activity of Smad protein increased by TGF-β was decreased by pheophorbide a in a concentration-dependent manner. As such, it was found that the phosphorylation of Smad activated through TGF-β signaling was inhibited by pheophorbide a.

### Experimental Example 3. Analysis of Anti-Fibrotic Activity of Pheophorbide a Compound

### Experimental Example 3.1. Analysis of Anti-Fibrotic Activity Using Western Blotting Assay

A cell-based assay experiment was performed using Western blotting assay to analyze the anti-fibrotic activity of pheophorbide a.

CCD8-Lu cells, which are human lung-derived fibroblasts, were cultured in the DMEM medium containing 10% FBS. Then, about 2×10⁵ cells were inoculated into a 6 well plate and cultured for 24 hours, and subjected to the deficient condition in the DMEM medium from which FBS was removed for about 24 hours.

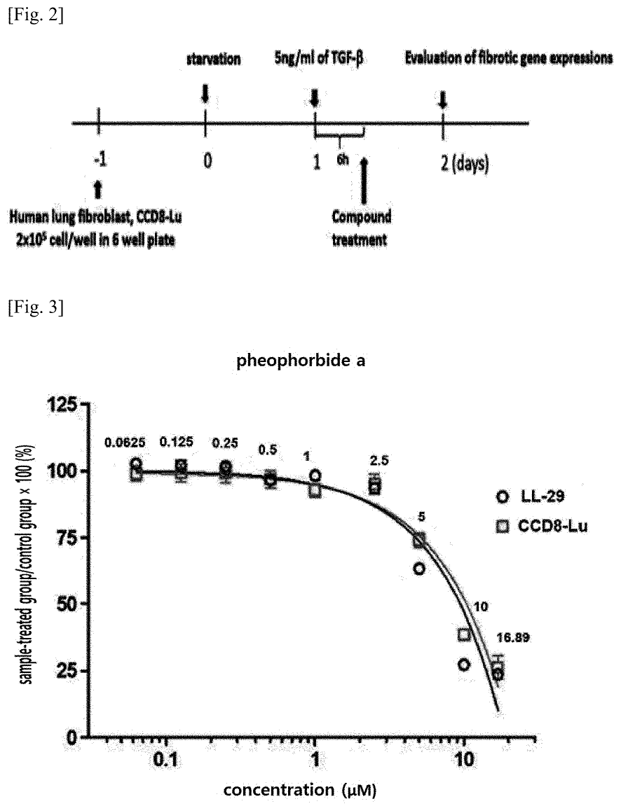

24 hours later, following the treatment with TGF-β at a concentration of 5 ng/ml, fibrosis of cells was induced for 6 hours. After 6 hours, the cells were treated with pheophorbide a obtained in Preparation Example 2 and further cultured for 18 hours (FIG. 2). Then, the negative control group was treated with nothing, the positive control group was treated with TGF-β alone at a concentration of 5 ng/ml, and the experimental groups were treated with TGF-β at a concentration of 5 mg/mL and 6 hours later, with pheophorbide a at each concentration of 0.06 μM, 0.12 μM, 0.25 μM, 0.5 μM, 2.5 μM and 5 μM.

After 18 hours, the culture medium was removed and the cells were washed twice by adding a PBS buffer solution, and 100 μl of RIPA buffer solution (150 mM NaCl, 1% NP-40, 0.5% Deoxycholic acid, 0.1% SDS, 50 mM Tris pH 7.5) containing a protease inhibitor was added thereto directly. 10 minutes later, the cells were recovered using a scraper and transferred to a microtube and centrifuged at about 12,000×g for 10 minutes.

The separated supernatant was obtained and transferred to a new microtube and then the protein was quantified using a bicinchoninic acid assay (BCA assay) protein quantitative kit. Each sample with the same amount of protein was subjected to electrophoresis, and then Western blotting was performed. Anti-collagen 1A or anti-fibronectin, anti-alpha-smooth muscle actin antibody were used as primary antibodies, and anti-mouse IgG HRP or an anti-rabbit IgG HRP were used as secondary antibodies. The difference in protein expression was corrected using anti-β-actin antibody for correction.

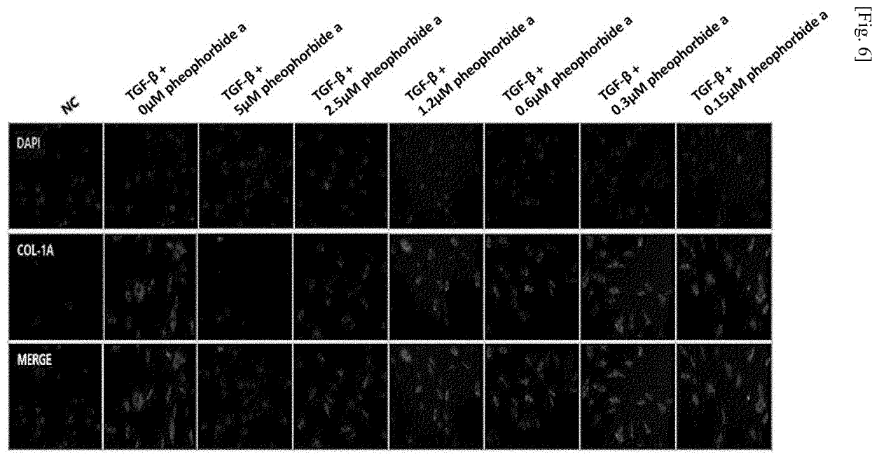

According to the experimental results shown in FIG. 5, it was found that the expression of collagen 1A, fibronectin, and smooth muscle actin was increased when treating CCD8-Lu cells which are human lung-derived fibroblasts with TGF-β, and it was confirmed that the expression of collagen 1A, fibronectin, and smooth muscle actin proteins induced by TGF-β was inhibited by treatment with pheophorbide a. As a result, pheophorbide a inhibited the expression of the protein characteristic of fibrosis, thereby confirming the possibility of suppressing the progression of fibrosis.

### Experimental Example 3.2. Analysis of Anti-Fibrotic Activity Using Immunocytochemistry Staining Assay

A cell-based assay experiment was performed to analyze the anti-fibrotic activity of pheophorbide a obtained in Preparation Example 2, using immunocytochemistry staining method.

CCD8-Lu cells, which are human lung-derived fibroblasts, were cultured in the DMEM medium containing 10% FBS. About 2×10⁴ cells were then inoculated into a 24 well plate and cultured for 24 hours, and subjected to the deficient condition in the DMEM medium from which FBS was removed for about 24 hours.

After 24 hours, following the treatment with TGF-β at a concentration of 5 ng/ml, fibrosis of cells was induced for 6 hours. After 6 hours, the cells were treated with pheophorbide a obtained in Preparation Example 2 and further cultured for 18 hours (FIG. 2). Then, the negative control group was treated with nothing, the positive control group was treated with TGF-β alone at a concentration of 5 ng/ml, and the experimental groups were treated with TGF-β at a concentration of 5 ng/ml and 6 hours later, with pheophorbide a at each concentration of 0.15 μM, 0.3 μM, 0.6 μM, 1.2 μM, 2.5 μM and 5 μM.

Immunohistochemistry analysis for each sample was performed as follows. First, the sample was washed twice with a PBS buffer, and then 4% formaldehyde was added to fix for 1 hour, and anti-collagen 1A was used as a primary antibody and Alexa Fluor 488 was used as a secondary antibody. The nuclei were stained using DAPI (4,6-diamidino-2-phenylindole) and the difference in the intensity of fluorescence was analyzed using a fluorescence microscope.

As a result of the experiment, as shown in FIG. 6, it was confirmed by the intensity of fluorescence that the expression of collagen 1A was low in the negative control CCD8-Lu cells and was significantly increased when treated with 5 ng/ml of TGF-β. In addition, it was confirmed that when treated with 5 ng/ml of TGF-β and 6 hours later, with pheophorbide a, the increased expression of collagen 1A induced by TGF-β was largely inhibited; and in particular, treatment with pheophorbide a at a concentration of 2.5 μM or more showed the most excellent inhibitory effect on the increased expression of collagen 1A induced by the TGF-β. As a result, it was found that the progression of fibrosis was inhibited by pheophorbide a.

### Experimental Example 4. Comparison of Anti-Fibrotic Activity Between Pheophorbide a and Therapeutic Agent for Pulmonary Fibrosis

### Experimental Example 4.1. Comparison of Anti-Fibrotic Activity Using Quantitative Real-Time Polymerase Chain Reaction

A cell-based analysis experiment was performed to analyze the anti-fibrotic activity of pheophorbide a obtained in Preparation Example 2 in comparison with nintedanib and pirfenidone, which were previously approved as a therapeutic agent for pulmonary fibrosis, using quantitative real-time polymerase chain reaction.

CCD8-Lu cells, which are human lung-derived fibroblasts, were cultured in the DMEM medium containing 10% FBS. About 2×10⁵ cells were then inoculated into a 6 well plate, cultured for 24 hours, and subjected to the deficient condition in the DMEM medium from which FBS was removed for about 24 hours.

24 hours later, following the treatment with TGF-β at a concentration of 5 ng/ml, fibrosis of cells was induced for 6 hours. After 6 hours, the cells were treated with pheophorbide a obtained in Preparation Example 2 or the therapeutic agent for pulmonary fibrosis and further cultured for 18 hours. Then, the negative control group was treated with nothing, the positive control group was treated with TGF-β alone at a concentration of 5 ng/ml, and the experimental groups were treated with TGF-β at a concentration of 5 ng/ml and 6 hours later, with pheophorbide a at a concentration of 2.5 μM or 5 nintedanib at a concentration of 1 μM, and pirfenidone at a concentration of 1 mM, respectively.

After 18 hours, the culture medium was removed, the cells were washed twice by adding a PBS buffer solution, and 0.5 ml of RNA extract (trizol reagent, Thermo Fisher Scientific) was directly added thereto, followed by being allowed to stand at room temperature for 10 minutes. Then, 0.1 ml of chloroform was added and stirred for 15 seconds, and then centrifuged at about 12,000×g for 10 minutes.

The supernatant was separated, added with the same volume of isopropyl alcohol and centrifuged at 12,000×g for 10 minutes. The liquid was then removed, and the resulting material was washed once with 75% ethanol and then dried at room temperature. After drying, about 50 μl of RNAase-free purified distilled water was added thereto, and the quantity and purity of the obtained RNA was measured using a spectrophotometer.

To synthesize cDNA using the obtained RNA, 2 μg of the total RNA purified was subjected to an anealing reaction with oligo dT at 70° C. for 5 minutes, and then 10× reverse transcription buffer solution, 10 mM dNTP, an RNAse inhibitor and M-MLV reverse transcriptase (Enzynomics, Korea) were added to perform cDNA synthesis reaction at 42° C. over 60 minutes.

After the cDNA synthesis reaction was completed, the reverse transcriptase was inactivated by heating at 72° C. for 5 minutes, and then RNase H was added to remove the single-stranded RNA and obtain the final cDNA. Whether the expression of the alpha-smooth muscle actin gene, which is a characteristic gene of myofibroblasts, the CCN2 (or CTGF) gene, which is a characteristic gene for fibrosis, fibronectin, which is a characteristics gene for fibrosis, and the NOX4 (NADPH oxidase 4) gene known as a major regulator of the amount of reactive oxygen species was changed was observed through quantitative real-time polymerase chain reaction. The GAPDH gene was quantified together to correct differences in expression. The nucleotide sequences of the genes used in the quantitative real-time polymerase chain reaction are shown in Table 2 below.

TABLE 2

| | | SEQ ID |
|---|---|---|
| Primer | Sequence | NO |
| alpha- | 5′-GCCCAGCCAAGCACTGTCAGGA-3′ | SEQ ID |
| SMA-S | | NO: 2 |
| alpha- | 5′-TCCCACCATCACCCCCTGATGTC-3′ | SEQ ID |
| SMA-AS | | NO: 3 |
| CTGF-S | 5′-GGCTTACCGACTGGAAGAC-3′ | SEQ ID |
| | | NO: 4 |
| CTGF- | 5′-AGGAGGCGTTGTCATTGG-3′ | SEQ ID |
| AS | | NO: 5 |
| Fibro- | 5′-GTGGCTGAAGACACAAGGAA-3′ | SEQ ID |
| nectin- | | NO: 6 |
| S | | |
| Fibro- | 5′-CCTGCCATTGTAGGTGAAT-3′ | SEQ ID |
| nectin- | | NO: 7 |
| AS | | |
| NOX4- | 5′-CACCTCTGCCTGTTCATCTG-3′ | SEQ ID |
| S | | NO: 8 |
| NOX4- | 5′-GGCTCTGCTTAGACACAATCC-3′ | SEQ ID |
| AS | | NO: 9 |
| GAPDH- | 5′-GTCTCCTCTGACTTCAACAGCG-3′ | SEQ ID |
| S | | NO: 10 |
| GAPDH- | 5′-ACCACCCTGTTGCTGTAGCCAA-3′ | SEQ ID |
| AS | | NO: 11 |

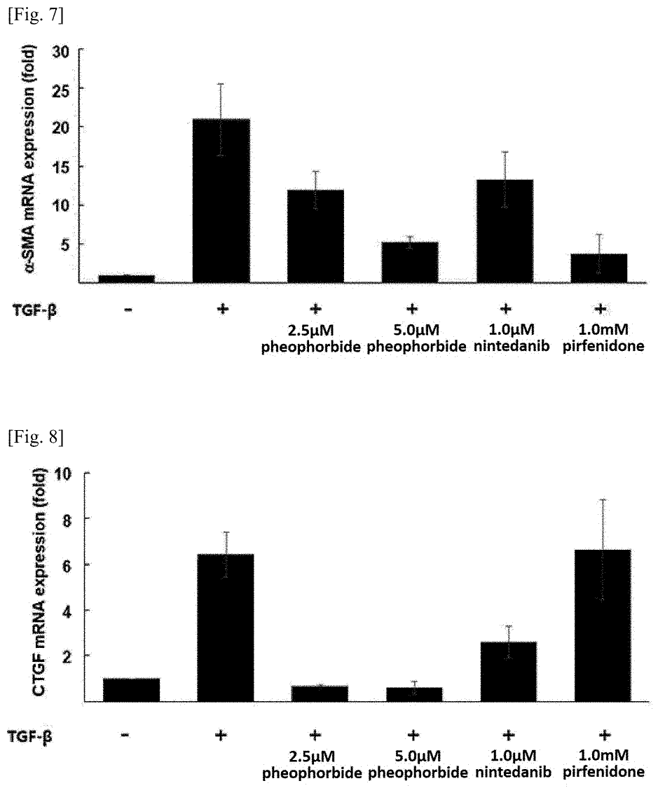

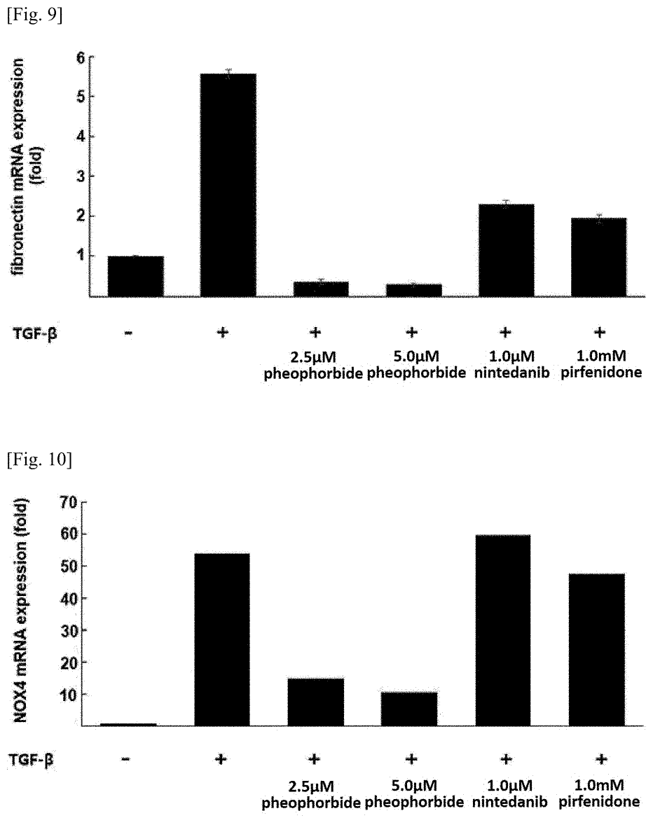

According to the experimental results shown in FIGS. 7 to 10, it was found that the expression of α-SMA, CTGF and NOX4 genes was increased when CCD8-Lu cells which are human lung-derived fibroblasts were treated with TGF-β, and it was confirmed that the treatment with pheophorbide a at a concentration of 2.5 μM and 5 μM remarkably inhibited the expression of the α-SMA, CTGF and NOX4 genes induced by TGF-β. On the other hand, it was confirmed that 1 μM nintedanib and 1 mM of pirfenidone weakly or hardly inhibited the expression of α-SMA, CTGF and NOX4 genes induced by TGF-β. As a result, it was confirmed that under the same conditions, pheophorbide a exhibited a remarkably excellent anti-fibrotic activity as compared with nintedanib and pirfenidone, the existing therapeutic agents for pulmonary fibrosis.

### Experimental Example 4.2. Comparison of Anti-Fibrotic Activity Based on Fibrosis-Related Protein Expression

A cell-based analysis experiment was performed to analyze the anti-fibrotic activity of pheophorbide a in comparison with nintedanib and pirfenidone, which were previously approved as a therapeutic agent for pulmonary fibrosis, using Western blotting assay.

CCD8-Lu cells, which are human lung-derived fibroblasts, were cultured in the DMEM medium containing 10% FBS. About 2×10⁵ cells were then inoculated into a 6 well plate, cultured for 24 hours, and subjected to the deficient condition in the DMEM medium from which FBS was removed for about 24 hours.

24 hours later, following the treatment with TGF-β at a concentration of 5 ng/ml, fibrosis of cells was induced for 6 hours. Then, the cells were treated with pheophorbide a obtained in Preparation Example 2 or the therapeutic agent for pulmonary fibrosis and further cultured for 18 hours. Then, the negative control group was treated with nothing, the positive control group was treated with TGF-β alone at a concentration of 5 ng/ml, and the experimental groups were treated with TGF-β at a concentration of 5 ng/ml and 6 hours later, with pheophorbide a at a concentration of 2.5 μNI or 5 μM, nintedanib at a concentration of 1 μM, and pirfenidone at a concentration of 1 mM, respectively.

After 18 hours, the culture medium was removed and the cells were washed twice by adding a PBS buffer solution, and 100 μl of RIPA buffer solution (150 mM NaCl, 1% NP-40, 0.5% Deoxycholic acid, 0.1% SDS, 50 mM Tris pH 7.5) containing a protease inhibitor was added thereto directly. 10 minutes later, the cells were recovered using a scraper and transferred to a microtube and centrifuged at about 12,000×g for 10 minutes.

The separated supernatant was obtained and transferred to a new microtube and then the protein was quantified using a bicinchoninic acid assay (BCA assay) protein quantitative kit. Each sample with the same amount of protein was subjected to electrophoresis, and then Western blotting was performed. Anti-α-SMA, anti-collagen 1A and anti-fibronectin were used as primary antibodies, and anti-mouse IgG HRP or an anti-rabbit IgG HRP were used as secondary antibodies. The difference in protein expression was corrected using an anti-β-actin antibody for correction.

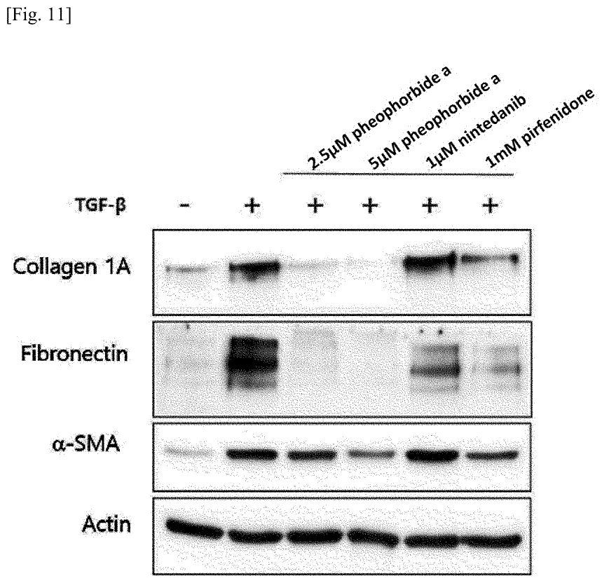

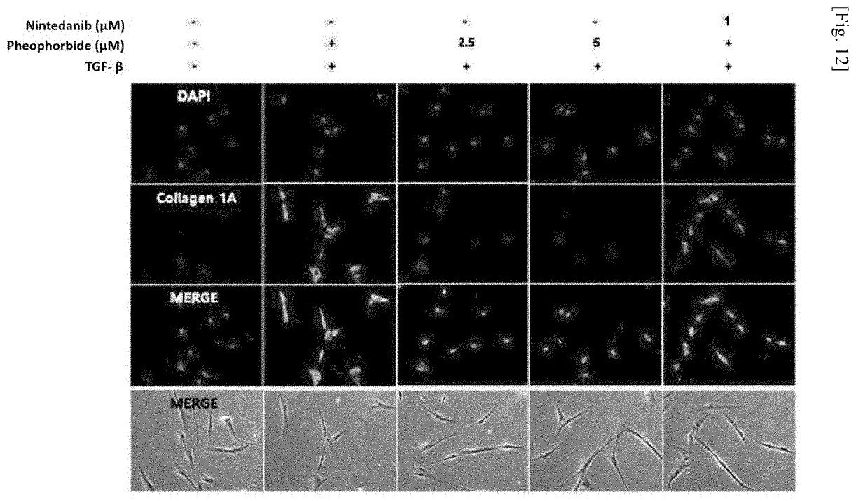

According to the experimental results shown in FIG. 11, it was found that the expression of α-SMA, collagen 1A, and fibronectin was increased when CCD8-Lu cells which are human lung-derived fibroblasts were treated with TGF-β, and it was confirmed that the treatment with pheophorbide a at a concentration of 2.5 μM and 5 μM remarkably inhibited the expression of the α-SMA, collagen 1A, and fibronectin induced by TGF-β. On the other hand, it was confirmed that 1 μM nintedanib and 1 mM of pirfenidone weakly or hardly inhibited the expression of α-SMA, collagen 1A, and fibronectin induced by TGF-β. As a result, it was confirmed that under the same conditions, pheophorbide a exhibited a remarkably excellent anti-fibrotic activity as compared with nintedanib and pirfenidone, the existing therapeutic agents for pulmonary fibrosis.

### Experimental Example 4.3. Analysis of Anti-fibrotic Activity Using Immunocytochemistry Staining Assay

A cell-based analysis experiment was performed to analyze the anti-fibrotic activity of pheophorbide a in comparison with nintedanib and pirfenidone, which were previously approved as a therapeutic agent for pulmonary fibrosis, using immunocytochemistry staining method.

CCD8-Lu cells, which are human lung-derived fibroblasts, were cultured in the DMEM medium containing 10% FBS. About 2×10⁴ cells were then inoculated into a 24 well plate and cultured for 24 hours, and subjected to the deficient condition in the DMEM medium from which FBS was removed for about 24 hours.

After 24 hours, following the treatment with TGF-β at a concentration of 5 ng/ml, fibrosis of cells was induced for 6 hours. After 6 hours, the cells were treated with 2.5 μM or 5 μM of pheophorbide a, 1 μM of nintedanib, or 1 mM of pirfenidone.

Immunohistochemistry analysis for each sample was performed as follows. First, the sample was washed twice with a PBS buffer, and then 4% formaldehyde was added to fix for 1 hour, and anti-collagen 1A was used as a primary antibody and Alexa Fluor 488 was used as a secondary antibody. The nuclei were stained using DAPI (4,6-diamidino-2-phenylindole) and the difference in the intensity of fluorescence was analyzed using a fluorescence microscope.

As a result of the experiment, as shown in FIG. 12, it was confirmed by the intensity of fluorescence that the expression of collagen 1A was low in the negative control CCD8-Lu cells and was significantly increased when treated with 5 ng/ml of TGF-β. In addition, it was confirmed that when treated with 5 ng/ml of TGF-β and 6 hours later, with pheophorbide a, the increased expression of collagen 1A induced by TGF-β was remarkably inhibited at a pheophorbide a concentration of 2.5 μM and 5 μM. On the other hand, it was confirmed that the treatment with 1 μM nintedanib weakly inhibited the expression of the collagen 1A protein induced by TGF-β. As a result, it was confirmed that under the same conditions, pheophorbide a exhibited a remarkably excellent anti-fibrotic activity as compared with nintedanib, the existing therapeutic agent for pulmonary fibrosis.

### Experimental Example 5. Confirmation of Expression Regulation of Phosphorylated Proteins Associated with Fibrosis

A cell-based assay experiment was performed using Western blotting assay to determine the effect of pheophorbide a on phosphorylation in the TGF-β signaling pathway.

A cell-based analysis experiment was performed to analyze the anti-fibrotic activity of pheophorbide a in comparison with nintedanib and pirfenidone, which were previously approved as a therapeutic agent for pulmonary fibrosis, using Western blotting assay.

CCD8-Lu cells, which are human lung-derived fibroblasts, were cultured in the DMEM medium containing 10% FBS. About 2×10⁵ cells were then inoculated into a 6 well plate, cultured for 24 hours, and subjected to the deficient condition in the DMEM medium from which FBS was removed for about 24 hours.

24 hours later, following the treatment with TGF-β at a concentration of 5 ng/ml, fibrosis of cells was induced for 6 hours. Then, the cells were treated with pheophorbide a obtained in Preparation Example 2 or the therapeutic agent for pulmonary fibrosis and further cultured for 18 hours. Then, the negative control group was treated with nothing, the positive control group was treated with TGF-β alone at a concentration of 5 ng/ml, and the experimental groups were treated with TGF-β at a concentration of 5 ng/ml and 6 hours later, with pheophorbide a at a concentration of 5 μM and nintedanib at a concentration of 1 μM, respectively.

After 1 hour, the culture medium was removed and the cells were washed twice by adding a PBS buffer solution, and 100 μl of RIPA buffer solution (150 mM NaCl, 1% NP-40, 0.5% Deoxycholic acid, 0.1% SDS, 50 mM Tris pH 7.5) containing a protease inhibitor was added thereto directly. 10 minutes later, the cells were recovered using a scraper and transferred to a microtube and centrifuged at about 12,000×g for 10 minutes.

The separated supernatant was obtained and transferred to a new microtube and then the protein was quantified using a bicinchoninic acid assay (BCA assay) protein quantitative kit. Each sample with the same amount of protein was subjected to electrophoresis, and then Western blotting was performed. An anti-pSmad3 antibody, an anti-Smad3 antibody, an anti-pERK antibody and an anti-ERK antibody were used as primary antibodies, and anti-mouse IgG HRP or an anti-rabbit IgG HRP were used as secondary antibodies.

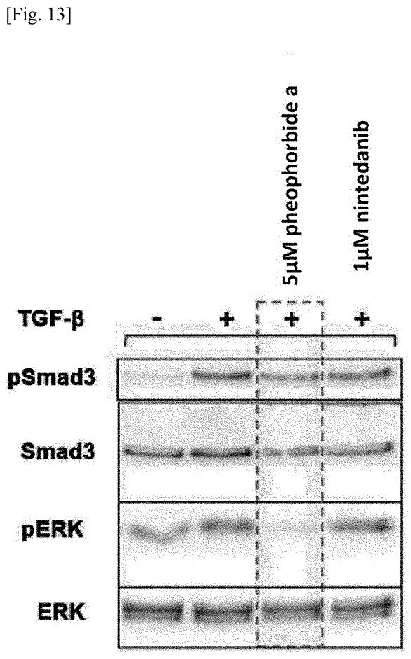

According to the experimental results shown in FIG. 13, it was found that the expression of phosphorylated Smad3 and phosphorylated ERK was increased when CCD8-Lu cells which are human lung-derived fibroblasts were treated with TGF-β, and it was confirmed that the treatment with pheophorbide a at a concentration of 5 μM remarkably inhibited the expression of phosphorylated Smad3 and phosphorylated ERK induced by TGF-β. On the other hand, it was confirmed that 1 μM nintedanib hardly inhibited the expression of phosphorylated Smad3 and phosphorylated ERK induced by TGF-β. As a result, it was confirmed that pheophorbide a exhibited an anti-fibrotic efficacy by inhibiting the expression of phosphorylated Smad3 and phosphorylated ERK proteins induced by TGF-β.

### Experimental Example 6. Identification of Anti-Fibrotic Effect of Pheophorbide a in Pulmonary Fibrosis Model Mice

In this experiment, the anti-fibrotic effect of pheophorbide a was evaluated in a pulmonary fibrosis model in which pulmonary fibrosis was induced by treating C57BL/6NCr10ri mice (Orient Bio) with bleomycin (BLM) (NIPPON KAYAKU, KIT code No. NA). The experimental groups were made as shown in Table 3 below.

TABLE 3

| | | | | | Oral | | | |
|---|---|---|---|---|---|---|---|---|
| | | Number | Instillation | BLM | Administration | Effective | | Nintedanib |
| | | of | Volume | dose | Volume | Substance | | Dose |
| Group | Sex | Animal | (μl) | (mg/kg) | (ml/kg) | (mg/kg) | | (mg/kg) |
| Vehicle control | Male | 5 | 50 | — | 10 | — | | — |
| Negative control | Male | 5 | 50 | 1.8 | 10 | — | | — |
| (Fibrosis control) | | | | | | | | |
| T1 | Male | 5 | 50 | 1.8 | 10 | 1.25 | mpk | — |
| T2 | Male | 5 | 50 | 1.8 | 10 | 5 | mpk | — |
| T3 | Male | 5 | 50 | 1.8 | 10 | 20 | mpk | — |
| T4 | Male | 5 | 50 | 1.8 | 10 | 80 | mpk | — |
| Positive control | Male | 5 | 50 | 1.8 | 10 | 60 | mpk | 60 |
| (Nintedanib) | | | | | | | | |

Mice were weighed and divided into total seven groups, and then administered intratracheally with sterile physiological saline in the vehicle control group and with BLM in the other groups than the vehicle control group on Day 1 to induce pulmonary fibrosis. Then, from Day 1 to Day 21, DMSO (sigma D2650-5X) devoid of the effective substance was administered to the vehicle control group and the negative control group (fibrosis control).

In addition, T1 to T4 groups and the positive control group were orally administered with the effective substance (pheophorbide a) and nintedanib, respectively, once daily. On day 1, however, the effective substance or the excipient was administered orally one hour before the BLM was administered intratracheally.

Following the administration, the death and the moribund status were observed once daily, and general symptoms such as appearance and behavioral changes were observed twice daily. Animals were observed in a broader range with an increased frequency when judged to be abnormal, and euthanized when toxicities comparable to severe affliction or death were shown. Body weight was measured 7 times in total (Day 1, 2, 4, 8, 11, 15 and 18) during the administration and once on the autopsy day.

On Day 22, the animals were euthanized by isoflurane inhalation and subjected to autopsy. First, the postcaval vein and the saphenous vein were cut to release blood to exsanguination, the presence of abnormalities in the appearance of thoracic cavity and abdominal cavity was observed, and then the lungs were extracted. The extracted lungs were weighed and a portion (left lung) thereof was fixed with 10% neutral buffered formalin solution for histopathological examination. A tissue specimen was prepared from the fixed lung, stained with hematoxylin&eosin (H&E) and subjected to histopathological examination. The organ weight ratio of the extracted lungs was calculated by using the following calculation formula.

Organ weight ratio=total lung weight(g)/total body weight(g) <Equation 1>

The results of the experiments were expressed by the mean and the standard deviation and statistically analyzed using Pristima System or Statistical Analysis System (SAS/STAT).

In the case of statistical analysis using the Pristima System, the comparison between groups was performed by multi-comparison analysis. The experimental data were subjected to homogeneity of variance test using Bartlett's Test, and then homoscedastic data was tested by One-Way Analysis of Variance (ANOVA) and the inter-group difference was analyzed by Dunnett's Test. The non-homoscedastic data was analyzed by Kruskal-Wallis Test, and the difference between the administration group and the control group was analyzed by Dunn's Rank Sum Test. Alternatively, the F-test was performed for the homogeneity of variance test between two groups.

In the statistical analysis using SAS/STAT, the homogeneity of variance test between two groups was performed using F-test. The homoscedastic data was subjected to Student's t-test to verify the inter-group difference, and the non-homoscedastic data was subjected to Wilcoxon Rank Sum Test. The statistical analysis methods of the experimental results are summarized in Table 4.

TABLE 4

| Parameters Tested | Methods of Statistical Analysis | | |
|---|---|---|---|
| Parameters Tested | Preliminary Test | Not significant | Significant |
| Body Weight | Bartlett's Test | ANOVA Test, | Kruskal-Wallis Test, |
| Body Weight Gain | F-test | Dunnett's Test | Dunn's Rank Sum Test |
| Organ Weight | | Student's t-test | Wilcoxon Rank Sum Test |

As a result, it was confirmed that in the pulmonary fibrosis model, the group to which pheophorbide a was administered showed an anti-fibrotic effect as compared with the negative control group. In addition, it was confirmed that the group to which pheophorbide a was administered showed a significant anti-fibrotic effect even as compared with the positive control group to which nintedanib was administered.

1.-7. (canceled)

8. A method of preventing, treating, or ameliorating fibrosis in a subject, comprising administering a therapeutically or nutritionally effective amount of a pheophorbide compound to the subject.

9. The method according to claim 8 wherein the pheophorbide compound is pheophorbide a.

10. The method according to claim 8, wherein the pheophorbide compound inhibits phosphorylation of Smad and ERK induced by TGF-β.

11. The method according to claim 8, wherein the pheophorbide compound inhibits the expression of alpha-smooth muscle actin, fibronectin or collagen induced by TGF-β.

12. The method according to claim 8, wherein the fibrosis is any one or more selected from the group consisting of hepatic fibrosis, pulmonary fibrosis, skin fibrosis, arthrofibrosis, nerve fibrosis, pancreatic fibrosis, muscle fibrosis, and peritoneal fibrosis.

13. The method according to claim 8 wherein the pheophorbide compound is comprised in a pharmaceutical composition.

14. The method according to claim 8 wherein the pheophorbide compound is comprised in a food composition.
